(19) United States
(12) Patent Application Publication
(10) Pub. No.: US 2011/0077232 A1
(43) Pub. Date: Mar. 31, 2011

(54) COMPOSITIONS COMPRISING ATORVASTATIN 4-(NITROOXY) BUTYL ESTER AND A HYPOLIPIDEMIC DRUG

(75) Inventors: Angela Monopoli, Milano (IT); Stefano Biondi, Pero (Milano (IT); Ennio Ongini, Milano (IT)

(73) Assignee: Nicox S.A., Sophia Antipolis - Valbonne (FR)

(21) Appl. No.: 12/995,953

(22) PCT Filed: May 18, 2009
(86) PCT No.: PCT/EP2009/055981

§ 371 Date: Dec. 2, 2010

(30) Foreign Application Priority Data
Jun. 6, 2008 (EP) 08157783.5

(51) Int. Cl. A61K 31/397; A61K 31/40; A61P 3/10; A61P 25/28; A61P 25/16; A61P 7/00; A61P 25/00; A61P 37/00

(52) U.S. Cl. 514/210.02; 514/423

(57) ABSTRACT

The present invention relates to compositions comprising atorvastatin 4-(nitrooxy) butyl ester and a hypolipidemic, in particular ezetimibe and fenofibrate. The invention discloses also their use as cholesterol-reducing drugs, as drugs having immunosuppressive properties, antioxidant, antithrombotic and anti-inflammatory activity, effects on endothelial function, and for treating and/or preventing acute coronary syndromes, stroke, atherosclerosis, neurodegenerative disorders, such as Alzheimer's and Parkinson's disease as well as autoimmune diseases, such as multiple sclerosis.

The present invention relates to compositions comprising atorvastatin 4-(nitrooxy) butyl ester (NO-atorvastatin) and a hypolipidemic drug, in particular ezetimibe and fenofibrate.

The invention discloses also their use as cholesterol-reducing drugs, as drugs having immunosuppressive properties, antioxidant, antithrombotic and anti-inflammatory activity, effects on endothelial function, and for treating and/or preventing acute coronary syndromes, stroke, atherosclerosis, neurodegenerative disorders, such as Alzheimer's and Parkinson's disease as well as autoimmune diseases, such as multiple sclerosis.

Hyperlipidemia, i.e. elevated levels of triglycerides or cholesterol, is a major cause of atherosclerosis and atherosclerosis-associated conditions, such as coronary heart disease, ischemic cerebrovascular disease and peripheral vascular disease.

Statins are the most effective and best tolerated drugs for treating hyperlipidemia. Statins are competitive inhibitors of HMG-CoA (3-hydroxy-3-methylglutaryl coenzyme A) reductase, an enzyme which catalyses an early, rate-limiting step in cholesterol biosynthesis. These drugs reduce triglyceride levels and are also indicated for raising high-density lipoprotein cholesterol (HDL-C) levels [P. O. Bonetti et al., European Heart Journal (2003) 24, 225-248].

Moreover, there are several classes of non-statin cholesterol blood level lowering agents currently in use for treating dyslipidemia such as niacin, bile acid sequestrants, fibric acid derivatives, inhibitors of microsomal triglyceride transport protein (MTP), dietary and biliary cholesterol absorption inhibitors, acyl CoA: cholesterol acyl transferase (ACAT) inhibitors.

Ezetimibe is an azetidione-based cholesterol absorption inhibitor that blocks the intestinal absorption of cholesterol, resulting in lowered plasma total cholesterol and low-density lipoprotein (LDL) levels.

Fenofibrate is a fibric acid derivative which acts on peroxisome proliferator-activated receptors α (PPAR α). It is mainly used to reduce cholesterol levels in patients at risk of cardiovascular disease. Like other fibrates, it reduces both LDL and very low density lipoprotein (VLDL) levels, as well as increasing high-density liporotein (HDL) levels and reducing triglyceride levels.

Now, it has been reported that fenofibrate has side-effects such as for example gastrointestinal disturbances, dermatological, musculoskeletal and neurological disorders (Martindale, Thirty-third edition, p. 889). The most common adverse drug reactions associated with ezetimibe therapy include headache and diarrhea.

WO 2004/105754 discloses the process for the preparation of atorvastatin 4-(nitrooxy) butyl ester as well as its therapeutic use.

Thus, it was an object of the present invention to find further drugs that are suitable for the treatment of the above mentioned diseases and which are more effective than the drugs currently employed in therapy.

In particular, it has been recognized that a composition comprising (a) atorvastatin 4-(nitrooxy) butyl ester and (b) a hypolipidemic drug exhibits a strong anti-inflammatory, antithrombotic and antiplatelet activity and can be employed for reducing cholesterol and triglycerides levels, for raising HDL-C levels and for treating or preventing acute coronary syndromes, stroke, peripheral vascular diseases such as peripheral ischemia and all disorders associated with endothelial dysfunctions such as vascular complications in diabetic patients and atherosclerosis. They should also be employed for treating neurodegenerative and autoimmune diseases, such as Alzheimer's and Parkinson's disease as well as multiple sclerosis.

It has been so surprisingly found that the combinations of the present invention have a better lipidic profile compared to NO-atorvastatin, and show most favorable effects on liver- and vessel wall-derived inflammation markers and pro-inflammatory cytokines.

Moreover, the combination as hereafter defined shows a better effect than the corresponding drugs alone which can lead to the reduction of dose of the hypolipidemic drug and consequently the risk of undesired side effects.

Accordingly, the present invention relates to a composition comprising:

(a) atorvastatin 4-(nitrooxy) butyl ester and
(b) a hypolipidemic drug selected from the group consisting of niacin, fibric acid derivatives, bile acid sequestrants, MTP inhibitors, dietary and biliary cholesterol absorption inhibitors, ACAT inhibitors, squalene synthase inhibitors, cholesterol ester transfer protein (CETP) inhibitors, cannabinoid-1 receptor blockers, apolipoprotein (apo) A-I, apoA-I-mimetic peptides, antisense drugs, peroxisome proliferators activated receptor (PPAR) agonists, thyroid receptor agonists and proprotein convertase subtilisin kexin type 9 (PCSK9) inhibitors.
The fibric acid derivatives include, for example, clofibrate, gemfibrozil, fenofibrate, ciprofibrate and bezafibrate.
The bile acid sequestrants include, for example, cholestyramine, colestipol and colesevelam.
The MTP inhibitors include, for example:
1) BMS-20138 which has the structure:

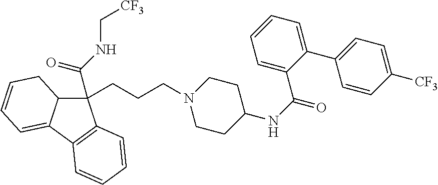

2) CP-346086 which has the structure:

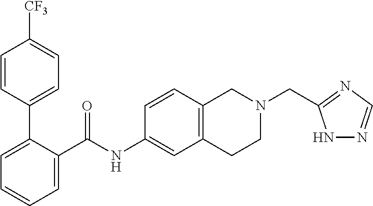

3) Implitamide which is (2S)-2-cyclopentyl-2-[4-[(2,4-dimethyl-9H-pyrido[2,3-b]indol-9-yl)methyl]phenyl]-N-[(1S)-2-hydroxy-1-phenylethylethanamide;
4) JTT-130 which is described in WO 03/072532 which is diethyl 2-(2-[3-dimethylcarbamoyl-4 [(4′ trifluoromethylbiphenyl-2-carbonyl)amino]phenyl]acetoxymethyl)-2-phenyl malonate; and
5) SLX 4090 which is [(3-methoxy-2-[(4-trifluoromethyl)phenyl]benzoyl)amino]-1,2,3,4-tetrahydro-2-isoquinolinecarboxylate.
The dietary and biliary cholesterol absorption inhibitors include, for example, ezetimibe.
The ACAT inhibitors include, for example:
1) avasimbe (CI-1011) which is sulfanic acid, [[2,4,6-tris(1-methylethyl)phenyl]acetyl]-,2,6-bis(1-methylethyl)phenyl ester;
2) F-1394 which is (1S,2S)-2-[3-(2,2-dimethylpropyl)-3-nonylureido]cyclohexane-1-yl3-[(4R)—N-(2,2,5,5-tetramethyl-1,3-dioxane-4-carbonyl)amino]propionate;
3) the azetidinone Sch 48461 which has the formula:

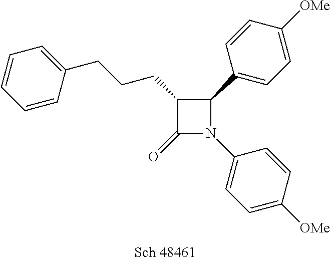

4) the azetidinone Sch 58053 which has the formula:

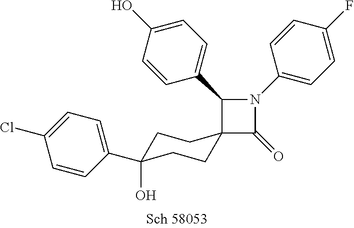

The squalene synthase inhibitors include, for example, lapaquistat.
The CETP inhibitors include, for example:

1) JTT-705 which has the formula:

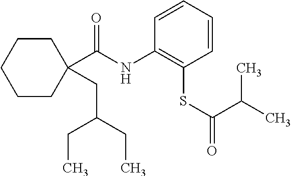

2) anacetrapib which has the formula:

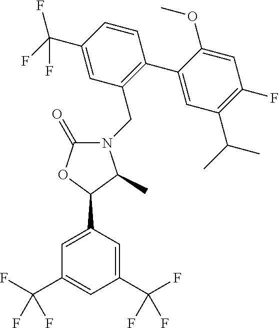

3) torcetrapib which has the formula:

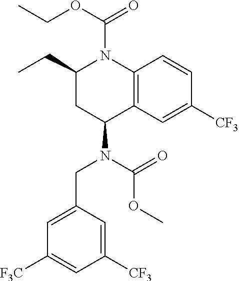

Cannabinoid-1 receptor blockers include, for example, rimonabant which has the formula:

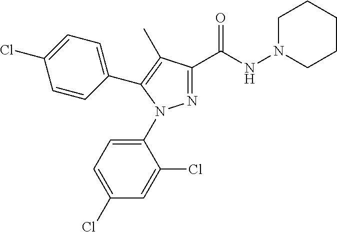

ApoA-I-mimetic peptides include, for example, D4F (Circulation 2004; 110:1701-1705).
Antisense drugs include, for example, apolipoprotein B-100 inhibitors such as Mipomersen.
PPAR alpha/gamma agonists include, for example, thiazolidinediones such as rosiglitazone, pioglitazone, troglitazone.
PCSK9 inhibitors include, for example, antisense oligonucleotide inhibitors (Journal of Lipid Research 48; 2007: 763-767).

The preferred compositions comprise:

(a) atorvastatin 4-(nitrooxy) butyl ester and
(b) a hypolipidemic drug selected from the group consisting of ezetimibe and fenofibrate.

The general synthesis of the NO-atorvastatin is described in the WO 2004/105754 (Example 7).

Both components (a) and (b) as part of the composition may be administered, simultaneously or sequentially, in their usual daily dosage or preferably in sub-effective doses.

In the composition according to the invention the amount of atorvastatin 4-(nitrooxy) butyl ester is in the range from 5 to 100 mg and the amount of ezetimibe or fenofibrate is in the range from 1 to 50 or from 10 to 200 mg, respectively.

The amount of the composition of the invention to be administered to the patient may vary depending on different factors well known to those skilled in the art, for example, the weight of the patient, the route of administration, or the severity of the illness.

Suitable pathways of administrations include but are not limited to oral, intravenous, intraperitoneal, transdermal, intrathekal, intramuscular, intranasal, transmucosal, subcutaneous, or rectal administration.

In a further aspect, the combinations of the present invention may be formulate with pharmaceutical acceptable eccipients according to the method known in the art.

### EXPERIMENTAL PART

### Method 1

Sixty female APOE*3Leiden mice (age 14-16 weeks, body weight 20-25 gram) were put on a semi-synthetic Western-type diet for weeks and were subsequently treated with or without NO-atorvastatin (4.3 mg/kg b.w. or 0.0036% w/w), ezetimibe (0.1 mg/kg b.w. or 0.000083% w/w), fenofibrate (1 mg/kg b.w. or 0.00083% w/w) and a combination of NO-atorvastatin with ezetimibe or fenofibrate for 4 weeks. After this period, the concentration of NO-atorvastatin in all groups treated with NO-atorvastatin and fenofibrate either alone or in combination was elevated three-fold (to respectively 13 mg/kg b.w. or 0.0108% w/w and 3 mg/kg b.w. or 0.0025% w/w). At the end of each treatment period blood was collected for the indicated lipid and inflammation parameters and at sacrifice livers and plasma/serum were collected.

APOE*3Leiden transgenic mice exhibit elevated plasma cholesterol and triglyceride levels, mainly confined to the VLDL/LDL sized lipoprotein fraction. This animal model has been proven to be representative for the human situation regarding plasma lipoprotein levels, lipoprotein profiles, its responsiveness to hypolipidemic drugs (like statins, fibrates etc.)

### Results

1.1. Plasma ALT levels

Alanine transaminase (ALT), as measure for liver function, was measured in pooled samples using the spectrophotometric assay of the Boehringer Reflotron system.

The data in Table 1 show that mice treated with fenofibrate alone showed increased ALT levels at t=4 weeks (+43%) and at t=8 weeks (+60%) as compared to the control mice. Combination of NO-atorvastatin with fenofibrate reversed the increase induced by fenofibrate alone. Moreover, combination treatment of NO-atorvastatin with fenofibrate or ezetimibe resulted in a reduction of ALT levels of 20% and 30%, respectively, compared to NO-atorvastatin treatment alone after 8 weeks.

### 1.2. Plasma Cholesterol

Total plasma cholesterol was determined using kit “Chol R1” is from Roche.

The data are reported in Table 2. NO-atorvastatin (−21% and −34%), ezetimibe (−25% and −35%), fenofibrate (no effect and −28%) NO-atorvastatin+ezetimibe (−46% and −59%) and NO-atorvastatin+fenofibrate (−34% and −60%) decreased plasma cholesterol levels after 4 and 8 weeks of treatment, respectively. Combination treatment of NO-atorvastatin with ezetimibe resulted in a reduction of plasma cholesterol levels of 32% and 38% compared to NO-atorvastatin treatment alone after 4 and 8 weeks, respectively. Combination treatment of NO-atorvastatin with fenofibrate resulted in a reduction of plasma cholesterol levels of 39% compared to NO-atorvastatin treatment alone after 8 weeks.

### 1.3. Plasma Triglycerides

Triglycerides level was determined using kit “Triglycerides GPO-PAP” from Roche.

The data in Table 3 show that treatment with fenofibrate decreased plasma triglycerides significantly compared to the control group at t=8 weeks by 41%. NO-atorvastatin in combination with fenofibrate decreased plasma triglycerides significantly by 50% as compared to NO-atorvastatin treatment alone after 8 weeks.

### 1.4. P-Selectin Levels

P-Selectin, adhesion molecule as inflammation marker, was determined using commercially available Elisa kit. After 8 weeks of treatment with NO-atorvastatin in combination with fenofibrate, the P-Selectin levels were decreased by 26% as compared to the NO-atorvastatin treated mice. Data are reported in Table 4.

### Method 2

Fifty-six female APOE*3Leiden mice (age 11-13 weeks, body weight 20-25 gram) were put on a semi-synthetic Western-type diet for 4 weeks and were subsequently treated with or without NO-atorvastatin (13.0 mg/kg b.w. or 0.0108% w/w), ezetimibe (0.1 mg/kg b.w. or 0.000083% w/w until week 6 and 0.3 mg/kg b.w. or 0.000249% w/w from week 6-12) and a combination of NO-atorvastatin with ezetimibe for 12 weeks. At time point t=0, t=4, t=8 and t=12 blood was collected for the measurement of the indicated lipid and inflammation parameters and at sacrifice serum, heart, thoracic aorta and liver were collected. The number of lesions and the number of undiseased segments in the aortic root were measured.

### Results

### 2.1. Number of Lesions

The number of lesions was significantly reduced in the NO-atorvastatin/ezetimibe combination group (with 56%, p<0.05) as compared to the control group and (with 47%, p<0.05) as compared to the NO-atorvastatin alone group. Treatment with NO-atorvastatin or ezetimibe alone did not influence the number of lesions as compared to the control group (Table 5). The number of lesions per cross section is presented as absolute values (means±SD).

### 2.2. Undiseased Segments

The data in Table 6 show that the number of undiseased segments was significantly increased in the NO-atorvastatin/ezetimibe combination group as compared to the control group (4.1-fold, p<0.001) and NO-atorvastatin alone group (2.0-fold, p=0.001). Treatment with ezetimibe did not affect the number of undiseased segments.

The number of undiseased segments in the aortic root was counted individually (n=12 per group) and calculated as percentage of all quantified segments. Values are percentages (means±SD).

The data in tables 5 and 6 show that the treatment with ezetimibe alone did not affect the progression of atherosclerosis. NO-atorvastatin alone reduced atherosclerosis in lesion area. The combination of NO-atorvastatin/ezetimibe reduced atherosclerosis more than the treatment with NO-atorvastatin alone, as reflected by the decrease in lesion number, the increase in the number of undiseased segments and tendencies towards decreased lesion area and relatively less severe lesions.

TABLE 1

| | ALT (U/L) | | |
|---|---|---|---|
| | t = 0 | t = 4 weeks | t = 8 weeks |
| Control | 120 | 117 | 127 |
| NO-atorvastatin | 120 | 114 | 91 |
| Ezetimibe | 120 | 92 | 81 |
| Fenofibrate | 120 | 167 | 203 |
| NO-atorvastatin + | 120 | 79 | 64 |
| Ezetimibe | | | |
| NO-atorvastatin + | 120 | 105 | 71 |
| Fenofibrate | | | |

Values are absolute values from measurements in pooled plasmas from n ≧ 9 mice per group.

TABLE 2

| | Cholesterol (mmol/L) | | |
|---|---|---|---|
| | t = 0 | t = 4 weeks | t = 8 weeks |
| Control | 18.6 ± 2.9 | 22.3 ± 2.9* | 22.9 ± 4.0* |
| NO-atorvastatin | 18.6 ± 2.8 | 17.7 ± 2.5* | 15.2 ± 3.0* |
| Ezetimibe | 18.6 ± 2.6 | 16.8 ± 2.4 | 14.9 ± 3.2* |
| Fenofibrate | 18.7 ± 2.2 | 21.4 ± 2.7 | 16.5 ± 2.2* |
| NO-atorvastatin + | 18.7 ± 2.2 | 12.1 ± 2.4*# | 9.4 ± 1.2*# |
| Ezetimibe | | | |
| NO-atorvastatin + | 18.6 ± 2.1 | 14.8 ± 1.6* | 9.2 ± 1.6*# |
| Fenofibrate | | | |

Values are absolute values and are means ± standard deviation (SD) of n ≧ 9 mice per group.
*p < 0.05 vs control;
#p < 0.05 vs NO-atorvastatin.

TABLE 3

| | Triglycerides (mmol/L) | | |
|---|---|---|---|
| | t = 0 | t = 4 weeks | t = 8 weeks |
| Control | 2.4 ± 0.6 | 2.3 ± 0.5 | 2.7 ± 1.0 |
| NO-atorvastatin | 2.7 ± 0.3 | 3.2 ± 0.7 | 2.7 ± 0.5 |
| Fenofibrate | 2.6 ± 0.7 | 2.2 ± 0.5 | 1.6 ± 0.5* |
| NO-atorvastatin + | 2.6 ± 0.4 | 2.7 ± 0.6 | 1.3 ± 0.3* |
| Fenofibrate | | | |

Values are absolute values and are means ± SD of n ≧ 9 mice per group.
*p < 0.05 vs control;
#p < 0.05 vs NO-atorvastatin.

TABLE 4

| | P-Selectin |
|---|---|
| | (ng/ml) |
| | t = 8 weeks |
| Control | 202 ± 37 |
| NO-atorvastatin | 190 ± 23 |
| Fenofibrate | 218 ± 39 |
| NO-atorvastatin + | 140 ± 31*# |
| Fenofibrate | |

Values are absolute values and are means ± SD of n ≧ 9 mice per group.
*p < 0.05 vs control;
#p < 0.05 vs NO-atorvastatin.

TABLE 5

| | Lesion number per |
|---|---|
| | cross section |
| | t = 12 weeks |
| Control | 3.8 ± 0.6 |
| NO-atorvastatin | 3.2 ± 0.7 |
| Ezetimibe | 3.5 ± 1.0 |
| NO-atorvastatin + | 1.7 ± 0.9*# |
| Ezetimibe | |

*p < 0.05 vs control;
#p < 0.05 vs NO-atrovastatin.

TABLE 6

| | Undiseased |
|---|---|
| | segments |
| | (% of total) |
| Control | 13 ± 11.8 |
| NO-atorvastatin | 26.4 ± 15.0 |
| Ezetimibe | 16.0 ± 14.8 |
| NO-atorvastatin + | 52.8 ± 18.9*# |
| Ezetimibe | |

*p < 0.05 vs control;
#p < 0.05 vs NO-atrovastatin.

1-20. (canceled)

21. A composition comprising:
(a) atorvastatin 4-(nitrooxy) butyl ester and
(b) a hypolipidemic drug.

22. A composition according to claim 21 wherein the hypolipidemic drug is selected from the group consisting of niacin, fibric acid derivatives, bile acid sequestrants, inhibitors of microsomal triglyceride transport protein (MTP), dietary and biliary cholesterol absorption inhibitors, acyl CoA: cholesterol acyl transferase (ACAT) inhibitors, squalene synthase inhibitors, cholesterol ester transfer protein (CETP) inhibitors, cannabinoid-1 receptor blockers, apolipoprotein (apo) A-I, apoA-I-mimetic peptides, antisense drugs, peroxisome proliferators activated receptor (PPAR) agonists, thyroid receptor agonists and proprotein convertase subtilisin kexin type 9 (PCSK9) inhibitors.

23. A composition according to claim 21 wherein the hypolipidemic drug is selected from the group consisting of fibric acid derivatives and dietary and biliary cholesterol absorption inhibitors.

24. A composition according to claim 23 wherein the hypolipidemic drug is selected from the group consisting of fenofibrate and ezetimibe.

25. A composition according to claim 24 wherein the amount of atorvastatin 4-(nitrooxy) butyl ester is in the range from 5 to 100 mg and the amount of ezetimibe is in the range from 1 to 50 mg.

26. A composition according to claim 24 wherein the amount of atorvastatin 4-(nitrooxy) butyl ester is in the range from 5 to 100 mg and the amount of fenofibrate is in the range from 10 to 200 mg.

27. A composition according to claim 21 for use as drug having anti-inflammatory, antithrombotic and antiplatelet activity.

28. A composition according to claim 21 for reducing cholesterol and triglycerides levels and/or for raising HDL-C levels.

29. A composition according to claim 21 for use in a method of treating acute coronary syndromes, stroke, peripheral vascular diseases and all disorders associated with endothelial dysfunctions.

30. A composition according to claim 29 for use in a method of treating peripheral ischemia, vascular complications in diabetic patients and atherosclerosis.

31. A composition according to claim 21 for use in a method of treating neurodegenerative and autoimmune disorders.

32. A composition according to claim 31 for use in a method of treating Alzheimer's disease, Parkinson's disease and multiple sclerosis.

33. A pharmaceutical composition comprising a combination according to claim 21 and pharmaceutically acceptable carriers.

34. A pharmaceutical composition according to claim 33 in a suitable form for the oral, intravenous, intraperitoneal, transdermal, intrathekal, intramuscular, intranasal, transmucosal, subcutaneous, or rectal administration.
